(19) United States
(12) Patent Application Publication
(10) Pub. No.: US 2003/0113923 A1
(43) Pub. Date: Jun. 19, 2003

(54) Steam-formaldehyde sterilization process indicator inks

(76) Inventor: Shobha Shakher Puntambekar, Edison, NJ (US)

Correspondence Address: Killworth, Gottman, Hagan & Schaeff, L.L.P., Suite 500, One Dayton Centre, Dayton, OH, 45402-2023, US

(21) Appl. No.: 10/021,694

(22) Filed: Dec. 12, 2001

(51) Int. Cl. G01N 33/00; G01N 21/78

(52) U.S. Cl. 436/8; 436/130; 436/164; 436/166; 436/181; 252/408.1

(57) ABSTRACT

Chemical indicator inks for steam-formaldehyde sterilization processes. The chemical indicator ink contains at least one primary organic dye selected from the group consisting of Congo red, Benzo purpurin B, Chicago sky blue 6B, Direct red 75, Evans blue, Naphthol blue black, Nitro red, and combinations thereof. The organic dye undergoes an irreversible color change when exposed to formaldehyde vapor in the presence of steam, but will not change color when exposed to other sterilization processes. The chemical indicator ink can further contain at least one secondary organic dye which does not undergo a color change when exposed to formaldehyde vapor in the presence of steam.

### BACKGROUND OF THE INVENTION

The present invention relates to steam-formaldehyde sterilization process indicators and, more particularly, to chemical indicator inks that are designed to undergo an irreversible color change when exposed to formaldehyde vapor in the presence of steam.

Biological and medical operations such as hospitals, medical laboratories, and other allied health facilities, which often come in contact with microorganisms and other microbiological agents, employ a variety of techniques to control both infection and contamination. While compositions such as germicides, antiseptics, and bacteriostats are effective in controlling widespread growth of biological contaminants, they do not go as far as to completely eliminate these agents. Consequently, steam-formaldehyde sterilization processes are often employed to ensure total eradication of microorganisms. The process generally involves placing items, for example, surgical instruments and non-sterile media, in a chamber and subjecting it to pulses of steam and formaldehyde vapor at sub-atmospheric pressure and relatively low temperature.

Known in the art are methods for indicating that an article has undergone steam-formaldehyde sterilization. See, e.g., U.S. Pat. No. 4,298,569 to Read. These methods employ chemical indicators which undergo an irreversible color change when placed in a steam-formaldehyde environment. However, the number of available color changes is limited. Thus, there is a need for chemical indicator inks which exhibit additional color changes upon exposure to formaldehyde vapor in the presence of low temperature steam.

### SUMMARY OF THE INVENTION

The present invention meets this need by providing chemical indicator inks for steam-formaldehyde sterilization processes. In accordance with one embodiment of the present invention, there is provided a chemical indicator ink which contains at least one primary organic dye selected from the group consisting of Congo red, Benzo purpurin B, Chicago sky blue 6B, Direct red 75, Evans blue, Naphthol blue black, Nitro red, and combinations thereof. The organic dyes will undergo an irreversible color change when exposed to formaldehyde vapor in the presence of steam. Moreover, the organic dyes will not undergo a color change when exposed to other sterilization processes such as ethylene oxide gas, high-temperature steam, dry heat, or combinations thereof. The concentration of the at least one primary organic dye can be between about 1.6 and about 23 g/l, and more typically between about 4.0 and about 8.0 g/l.

The chemical indicator ink of the present invention can further contain a stabilizing buffer. The stabilizing buffer typically has a pH of between about 4 and about 6. Preferably, the stabilizing buffer does not react with the at least one primary organic dye.

In accordance with another embodiment of the present invention, the chemical indicator ink further contains at least one secondary organic dye. The at least one secondary organic dye enhances the color change of the chemical indicator ink and does not undergo a color change when exposed to formaldehyde vapor in the presence of steam. The at least one secondary organic dye can be selected from the group consisting of methylene blue, trypan blue, direct blue 71, and combinations thereof.

The chemical indicator ink can be an aqueous ink solution. The aqueous ink solution comprises the at least one primary organic dye dissolved or dispersed in water.

The chemical indicator ink can further contain a thickening agent selected from the group consisting of waxes, gums, polymers, methyl cellulose, ethyl cellulose, carboxy methyl cellulose, and combinations thereof. In addition, the chemical indicator ink can further contain brighteners, binders, stabilizers, and preservatives.

### DETAILED DESCRIPTION OF THE INVENTION

The present invention provides a chemical indicator ink for steam-formaldehyde sterilization processes. The chemical indicator ink contains at least one primary organic dye. The at least one primary organic dye will undergo a visual, distinct, and irreversible color change when exposed to formaldehyde vapor in the presence of low-temperature steam. Accordingly, the chemical indicator ink composition of the present invention can be applied to a substrate to form a steam-formaldehyde sterilization process indicator, which is effective in signaling whether an article has been subjected to steam-formaldehyde sterilization processing.

Unlike biological indicators which utilize dried microbes and can be cultured in sterile media following processing to ensure sterilization, the chemical indicator ink of the present invention merely shows that an article has undergone steam-formaldehyde sterilization processing. It does not indicate whether any microbial contaminants were effectively destroyed. Consequently, it is important for the operator of the steam-formaldehyde sterilization process to follow correct sterilization protocols.

The at least one primary organic dye of the present invention is an organic compound which can be present as a free acid or one of its salts. Typically, the at least one primary organic dye has at least one azo linkage, at least one primary or secondary amino group, at least one sulfonic acid group, and at least one naphthalene residue, and can be selected from the group consisting of Congo red, Benzo purpurin B, Chicago sky blue 6B, Direct red 75, Evans blue, Naphthol blue black, Nitro red, and combinations thereof.

In accordance with one embodiment of the present invention, the at least one primary organic dye can be Congo red (cas. #573-58-6) or 3,3′-[4-4′-bisphenylylene-(azo)]bis[4-amino-1-naphthalene]. The chemical structure for this primary organic dye is:

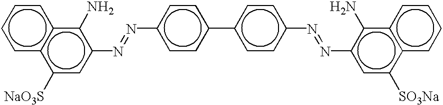

In accordance with another embodiment of the present invention, the at least one primary organic dye can be Benzo purpurin B (cas. #992-59-6) or 3,3′-[(3,3′-Dimethyl[1,1′-biphenyl]-4,4′-diyl)bis(azo)]bis[4-amino-1-naphthalenesulfonic acid]disodium salt. The chemical structure for this primary organic dye is:

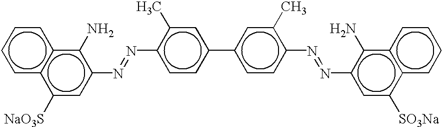

In accordance with another embodiment of the present invention, the at least one primary organic dye can be Chicago sky blue 6B (cas. #2610-05-01) or 6,6′-[(3,3-Dimethoxy[1,1′-biphenyl]-4,4′-diyl)bis(azo)]bis[4-amino-5-hydroxy-1,3-naphthalenedisulphonic acid]tetrasodium salt. The chemical structure for this primary organic dye is:

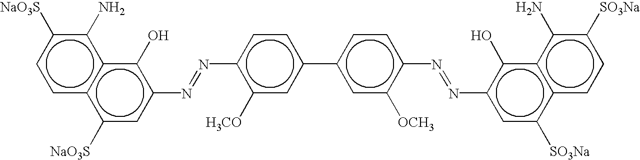

In accordance with another embodiment of the present invention, the at least one primary organic dye can be Direct red 75 (cas. #2829-43-8) or 2-Naphthalenesulfonic acid, 5,5′-[carbonylbis[imino(2-sulfo-4,1-phenylene)azo]]bis[6-amino-4-hydroxy, tetrasodium salt. The chemical structure for this primary organic dye is:

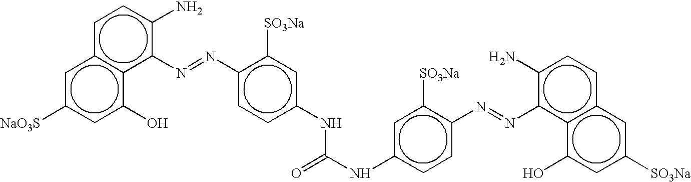

In accordance with another embodiment of the present invention, the at least one primary organic dye can be Evans blue (cas. #314-13-6) or 6,6′-[3,3′-Dimethyl(1,1-biphenyl)-4,4′-diyl}bis(azo)-bis-(4-amino-5-hydroxy)-1,3-naphthalenedisulfonic acid, tetrasodium salt. The chemical structure for this primary organic dye is:

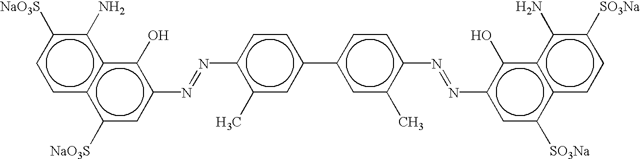

In accordance with another embodiment of the present invention, the at least one primary organic dye can be Naphthol blue black (cas. #1064-48-8) or 4-amino-5-hydroxy-3,3[(4-nitrophenyl)azo]-6-phenylazo)-2,7-naphthalene-Disulfonic acid disodium salt. The chemical structure for this primary organic dye is:

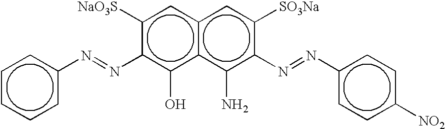

In accordance with another embodiment of the present invention, the at least one primary organic dye can be Nitro red (cas. #56431-61-9) or 2,7-Naphthalenedisulfonic acid, 4-amino-5-hydroxy-3-[(4-nitrophenyl)azo]-, disodium salt. The chemical structure for this primary organic dye is:

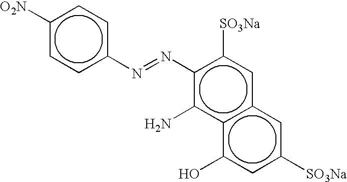

Each of the above-referenced primary organic dyes when subjected to formaldehyde vapor at about 70° C., exhibit distinct and irreversible color changes. The color changes are visually very marked. Consequently, it is readily apparent when viewing the steam-formaldehyde sterilization process indicator of the present invention whether the materials associated therewith have been subjected to steam-formaldehyde sterilization processing. Moreover, because each primary organic dye exhibits its own distinct color change, the present invention provides chemical indicator inks with additional color changes not contemplated by the prior art. The colors of the primary organic dyes of the present invention both before and after exposure to formaldehyde vapor are set forth in Table 1 below.

TABLE 1
Primary Organic Dyes Before and After Exposure to
Formaldehyde Vapor

| Primary Dye | Color Before Exposure | Color After Exposure |
|---|---|---|
| Congo red | Red | Yellow |
| Benzo purpurin | Red | Cream |
| Chicago sky blue 6B | Blue | Grayish Black |
| Direct red 75 | Magenta | Pale pink |
| Evans blue | Blue | Chocolate brown |
| Naphthol blue black | Blue | Grayish blue |
| Nitro red | Reddish purple | Pink |

In addition to the at least one primary organic dye, the chemical indicator ink of the present invention can include a buffer. Particularly useful buffer solutions are ones which do not react with the organic dye and which will buffer the organic dye to a desired pH range (i.e., potassium hydrogen phthalate buffer). The buffer acts to stabilize the organic dye until it is exposed to the steam-formaldehyde sterilization process. In addition, the presence of a buffer in the chemical indicator ink of the present invention appears to make the color change of the organic dye largely independent of the concentration of formaldehyde. This is particularly important when a relatively low concentration of formaldehyde is used.

The at least one primary organic dye is less sensitive to formaldehyde under alkaline conditions than it is under acid conditions. Consequently, a buffer with a pH of between about 4 and about 6 is particularly useful. At this pH range, although low formaldehyde vapor concentrations will bring about the desired color change, there is no color change when the chemical indicator ink is subjected to other types of sterilization processes (i.e., ethylene oxide gas, high-temperature steam, dry heat, or combinations thereof). If the pH of the buffer is below 4, the chemical indicator ink of the present invention will still react to low-temperature steam-formaldehyde sterilization. However, at this pH level, it may also react to ethylene oxide gas.

In addition to the at least one primary organic dye which changes color under steam-formaldehyde sterilizing conditions, the chemical indicator ink of the present invention can further include at least one secondary organic dye, which does not change color during steam-formaldehyde sterilization processing. The at least one secondary organic dye can be any color and is useful in enhancing the color change of the chemical indicator ink of the present invention. More particularly, the at least one secondary organic dye can be a blue dye selected from the group consisting of methylene blue, trypan blue, direct blue 71, and combinations thereof, which are then combined with the at least one primary organic dye.

In accordance with another embodiment of the present invention, the at least one secondary organic dye can be Direct blue 71 (cas #4399-55-7) or 1,5-Naphthalenedisulfonic acid, 3-[[4-[(6-amino-1-hydroxy-3-sulfo-2-naphthalenyl)azo]-6-sulfo-1-naphthalenyl]azo]-1-naphthalenyl]azo]-, tetrasodium salt. The chemical structure for this primary organic dye is:

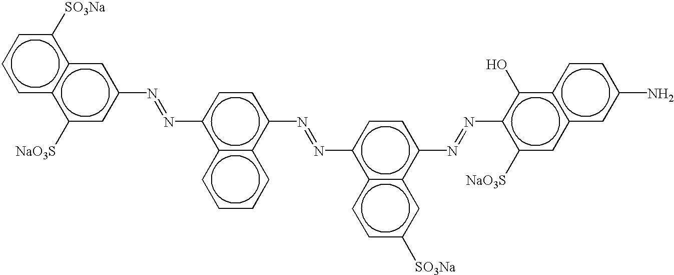

In accordance with another embodiment of the present invention, the at least one secondary organic dye can be Trypan blue (cas. #72-57-1) or 3,3′-{[3,3′-Dimethyl(1,1′-biphenyl)-4,4′-diyl]bis(azo)}bis-(5-amino-4-hydroxy)-2,7-naphthalenedisulfonic acid, tetrasodium salt. The chemical structure for this primary organic dye is:

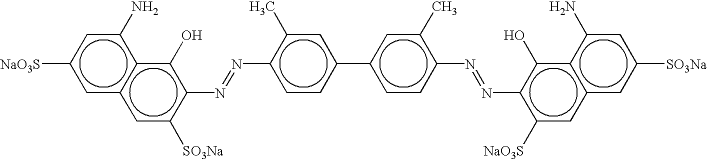

By using the at least one secondary organic dye in combination with the at least one primary organic dye, it is possible to choose the desired initial and final colors of the chemical indicator ink. This may be advantageous when distinguishing the steam-formaldehyde sterilization process indicator of the present invention from indicators which are used for other types of sterilization and to give a marked color change. The colors of the different primary and secondary organic dyes when combined in the presence of a buffer solution both before and after exposure to formaldehyde vapor are set forth in Table 2 below.

TABLE 2
Combined Primary and Secondary Organic Dyes Before and After
Exposure to Formaldehyde Vapor

| | | Color Before | Color After |
|---|---|---|---|
| Primary Dyes | Secondary Dyes | Exposure | Exposure |
| Congo red | +Methylene blue | Red | Green |
| +Buffer solution | +Direct blue 71 | Reddish purple | Gray |
| | +Trypan blue | Reddish purple | Gray |
| Benzo purpurin | +Methylene blue | Reddish purple | Blue |
| +Buffer solution | +Direct blue 71 | Reddish purple | Blue |
| | +Trypan blue | Reddish purple | Blue |
| Chicago sky blue | +Methylene blue | Blue | Grayish black |
| 6B +Buffer | +Direct blue 71 | Blue | Grayish black |
| solution | +Trypan blue | Blue | Gray |
| Direct red 75 | +Methylene blue | Magenta | Pale purple |
| +Buffer solution | +Direct blue 71 | Magenta | Grayish purple |
| | +Trypan blue | Purple | Grayish purple |
| Evans blue | +Methylene blue | Blue | Brown |
| +Buffer solution | +Direct blue 71 | Blue | Brown |
| | +Trypan blue | Blue | Grayish black |
| Naphthol blue black | +Methylene blue | Blue | Purple |
| +Buffer solution | +Direct blue 71 | Blue | Purple |
| | +Trypan blue | Blue | Grayish blue |

The concentration of the organic dye is typically between about 1.6 and about 23 g/l, and more typically between about 4.0 and about 8.0 g/l. The chemical indicator ink of the present invention can be an aqueous ink system, wherein the organic dye is dissolved or dispersed in water.

The chemical indicator ink can further include a thickening agent selected from the group consisting of waxes, gums, polymers, methyl cellulose, ethyl cellulose, carboxy methyl cellulose, and combinations thereof. Additional agents, i.e., gelatin, can also be added to the chemical indicator ink as brighteners. Gelatin, as well as other polymeric materials such as polyvinyl alcohol, can also assist in binding and stabilizing the chemical indicator ink until use. Moreover, the chemical indicator ink can further include a preservative such as sorbic acid (cas #110-44-1) or 2,4-Hexadienoic acid.

In order that the invention may be more readily understood, reference is made to the following example, which is intended to illustrate the invention, but not limit the scope thereof.

The chemical indicator ink used in the following example was prepared as follows:

1. 6 g gelatin (225 bloom) was dissolved in 100 ml water at 60° C.;

2. 34 g polyvinyl alcohol (PVA) was dissolved in 200 ml water at 80° C.;

3. 5 g organic dyes were dissolved in a mixture of 65 ml water and 65 ml isopropanol; and

4. the gelatin and polyvinyl alcohol solutions were added to the organic dyes solutions with stirring.

The PVA solution was prepared as follows:

Item 1. water (cas #7732-18-5)

Item 2. Elvanol® 51-05 polyvinyl alcohol (available from DuPont Company, Charlotte, N.C.)

Item 3. Dowicil™ 200 preservative (available from Dow Chemical Company, Midland, Mich.)

Add Item 1. Water should be heated to 120° C. Start mixer and add Item 2. Add Item 3 and mix until polyvinyl alcohol is dissolved.

The color change properties of the inks were tested as follows:

1. steam-formaldehyde ink 8 g

2. 5% potassium hydrogen phthalate buffer solution 0.705 g

3. 5% secondary organic dyes (table 2) aqueous solution 0.705 g

Portions of the resulting chemical indicator inks were tested by exposing to formaldehyde vapor in the presence of steam.

Further portions of the indicator inks were subjected to a steam autoclave sterilization in the absence of formaldehyde during which the temperature was 134° C. for 3.5 minutes. There was no color change. Further portions of the indicator inks were subjected to a dry heat at 140° C. for 30 minutes. There was no color change.

While certain representative embodiments and details have been shown for purposes of illustrating the invention, it will be apparent to those skilled in the art that various changes in the compositions and methods disclosed herein may be made without departing from the scope of the invention, which is defined in the appended claims.

What is claimed is:

1. A chemical indicator ink for steam-formaldehyde sterilization processes containing at least one primary organic dye selected from the group consisting of Congo red, Benzo purpurin B, Chicago sky blue 6B, Direct red 75, Evans blue, Naphthol blue black, Nitro red, and combinations thereof.

2. The chemical indicator ink of claim 1 wherein said at least one primary organic dye undergoes an irreversible color change when exposed to formaldehyde vapor in the presence of steam.

3. The chemical indicator ink of claim 2 wherein said steam has a temperature of about 70° C.

4. The chemical indicator ink of claim 1 wherein said at least one primary organic dye does not undergo a color change when exposed to ethylene oxide gas, high-temperature steam, dry heat, or combinations thereof.

5. The chemical indicator ink of claim 1 wherein the concentration of said at least one primary organic dye is between about 1.6 and about 23 g/l.

6. The chemical indicator ink of claim 5 wherein the concentration of said at least one primary organic dye is between about 4.0 and about 8.0 g/l.

7. The chemical indicator ink of claim 1 wherein said at least one primary organic dye is more sensitive to formaldehyde under alkaline conditions.

8. The chemical indicator ink of claim 1 wherein said at least one primary organic dye is less sensitive to formaldehyde under acidic conditions.

9. The chemical indicator ink of claim 1 further containing a stabilizing buffer.

10. The chemical indicator ink of claim 9 wherein said stabilizing buffer has a pH of between about 4 and about 6.

11. The chemical indicator ink of claim 9 wherein said stabilizing buffer does not react with said at least one primary organic dye.

12. The chemical indicator ink of claim 1 further containing at least one secondary organic dye.

13. The chemical indicator ink of claim 12 wherein said at least one secondary organic dye enhances the color change of said chemical indicator ink and does not undergo a color change when exposed to formaldehyde vapor in the presence of steam.

14. The chemical indicator ink of claim 12 wherein said at least one secondary organic dye is selected from the group consisting of methylene blue, trypan blue, direct blue 71, and combinations thereof.

15. The chemical indicator ink of claim 1 wherein said chemical indicator ink is an aqueous ink solution.

16. The chemical indicator ink of claim 15 wherein said aqueous ink solution comprises said at least one primary organic dye dissolved or dispersed in water.

17. The chemical indicator ink of claim 1 further containing a thickening agent and wherein said thickening agent is selected from the group consisting of waxes, gums, polymers, methyl cellulose, ethyl cellulose, carboxy methyl cellulose, and combinations thereof.

18. The chemical indicator ink of claim 1 further containing a brightener.

19. The chemical indicator ink of claim 18 wherein said brightener is gelatin.

20. The chemical indicator ink of claim 1 further containing a binder or stabilizer.

21. The chemical indicator ink of claim 20 wherein said binder or stabilizer is a polymeric material.

22. The chemical indicator ink of claim 21 wherein said polymeric material is polyvinyl alcohol.

23. The chemical indicator ink of claim 20 wherein said binder or stabilizer is gelatin.

24. The chemical indicator ink of claim 1 further containing a preservative.

25. The chemical indicator ink of claim 24 wherein said preservative is sorbic acid.
